(19) United States
(12) Patent Application Publication
(10) Pub. No.: US 2026/0358326 A1
(43) Pub. Date: Nov. 12, 2026

(54) ELECTROCHEMICAL METHOD OF TESTING OXIDATIVE STRESS

(71) Applicant: B.G. NEGEV TECHNOLOGIES AND APPLICATIONS LTD., AT BEN-GURION UNIVERSITY, Beer Sheva (IL)

(72) Inventor: Hadar Shmuel BEN-YOAV, Ramat Gan (IL)

(73) Assignee: B.G. NEGEV TECHNOLOGIES AND APPLICATIONS LTD., AT BEN-GURION UNIVERSITY, Beer Sheva (IL)

(21) Appl. No.: 19/117,765

(22) PCT Filed: Oct. 3, 2023
(86) PCT No.: PCT/IL2023/051057

§ 371 Date: Apr. 2, 2025

Related U.S. Application Data
(60) Provisional application No. 63/412,825, filed on Oct. 3, 2022.

(51) Int. Cl. G01N 33/487; G01N 27/30; G01N 27/48

(52) U.S. Cl. CPC G01N 33/48707; G01N 27/30; G01N 27/48

(57) ABSTRACT

A method of determining oxidative stress in a subject, comprising: obtaining a biofluid sample from the subject; acquiring an electrochemical signal from the sample with the aid of an array of bare and surface-modified electrodes that is sensitive towards a model antioxidant and a model oxidant; optionally preprocessing the electrochemical signal, to obtain processed data; applying trained chemometric model(s) to the processed or raw data, to measure TAG and TOS levels; and determining a status of oxidative stress in the subject based on the measured TAG, TOS or the TOS:TAG ratio.

### BACKGROUND OF THE INVENTION

Reactive oxygen species (ROS), such as the superoxide anion radical, the hydroxyl radical and hydrogen peroxide are formed normally as by-products of physiological processes but can be generated excessively under pathophysiological conditions. Antioxidants (substances that at low concentration can inhibit oxidation of oxidizable substrates) provide the defense against the deleterious action of such reactive oxygen species, i.e., non-enzymatic and enzymatic antioxidants. An imbalance between oxidants and antioxidants in favor of the oxidants is defined as ‘oxidative stress’ [Helmut Sies, Experimental Physiology (1997), 82, 291-295]. That is, oxidative stress is a situation where the balance has been disrupted such that the ability of the antioxidants to protect the body by removing the oxidizing species has become insufficient.

Urinalysis is a common method for detecting various diseases and conditions in a person. Oxidative stress is potentially detectable through urinalysis. Determining a state of oxidative stress in a person, by testing a urine sample for the presence of a specific biological marker of oxidative stress, such as malondialdehyde, was achieved by colorimetric assays, with the aid of suitable chemical reagents added to the urine sample (to produce a colored reaction product, namely, a measurable chromogen). Thus, the colorimetric change in the urine sample is indicative of the concentration of the malondialdehyde. See for example, U.S. Pat. No. 6,165,797 (where the reagent was based on basic fuchsin) and U.S. Pat. No. 8,198,097 (where detection of malondialdehyde was obtained with thiobarbituric acid).

CA 2331953 shows measurements of overall oxidative stress from a range of oxidative species (H₂O₂ as well as organic peroxides), by adding to a biofluid sample a reagent consisting of FeSO₄, 2-deoxyglucose, EDTA and thiobarbituric acid.

A different approach is shown in WO 2005/052575, where it was proposed to measure molecular markers indicative of oxidative stress with nuclear magnetic resonance (NMR) spectroscopy, infrared (IR) spectroscopy or chromatographic techniques.

Instead of quantifying a specific biomarker, it is possible to get a useful oxidative stress index by the ratio TOS:TAC, i.e., the ratio between the total oxidant status (TOS) and total antioxidant capacity (TAC) measured in a biofluid sample. A TOS:TAC ratio higher than a known threshold can serve as indication of oxidative stress.

Regarding TAC measurements, a recently published review about measurements of antioxidant activity [Munteanu et al., International Journal of Molecular Sciences 2021, 22, 3380] shows that the acceptable analytical methods fall into three categories: spectrophotometric, electrochemical and chromatographic techniques. Spectroscopy based on colorimetric assays seems to be the prevalent technique (see Table 2 of Munteanu et al.). For example, one common colorimetric assay is known as the ABTS test [2,2′-Azino-bis(3-ethylbenzothiazoline-6-sulfonic acid; available as the diammonium salt]. ABTS is colorless, but it can form a stable, blue-green radical cation, ABTS^{⋅+}, which in turn reacts with antioxidants. Hence, the characteristic color of ABTS^{⋅+}-containing solution gradually vanishes in the presence of antioxidants. The degree of discoloration of ABTS^{⋅+} solution (quantifiable as a decrease in the intensity of absorbance measured at wavelength of 600 nm) can be used to estimate TAC.

Regarding TOS measurements, again, spectrophotometric techniques seem to prevail [Rubio et al., BMC Veterinary Research (2021) 17:226]. An acceptable colorimetric assay for determination of TOS is based on the ferrous ion/o-dianisidine complex. Oxidants present in a sample oxidize the ferrous ion (Fe²⁺) to ferric ion (Fe³⁺). A colored complex is formed between the Fe³⁺ ion and xylenol orange, which absorbs in the range between 540 to 580 nm. Color intensity is therefore proportional to the concentration of oxidants in the test sample. The complex-formation reaction takes place in an acidic environment, in the presence of glycerol. The results are expressed as micromolar H₂O₂ equivalents per liter.

Unlike the spectrophotometric methods described above, which require two distinct colorimetric assays to measure TAC and TOS separately, electrochemical technique could potentially determine both variables simultaneously. However, electrochemical methods are not widely used in measuring TAC and TOS. See a mini review by Rey et al. [Biomedical Journal of Scientific & Technical Research, 11(2)-2018 (8376-8378)]. It seems that also in the patent literature, little has been reported on evaluation of oxidative stress by electrochemical techniques. In US 2009/0004686, the oxidative status was evaluated by measuring oxidation-reduction potential (ORP) with commercial redox electrode immersed in blood samples. US 2022/0031230 shows the determination of status of oxidative stress by measuring quantities called ‘total oxidizing power’ and ‘total antioxidizing power’ using microelectrodes attached to the skin. A gel comprising a compound that dissociates into an oxidizing form and an oxidant form is spread over a small area of the skin and an electrochemical versus time plot is recorded from which the abovementioned quantities are derived. The authors indicate that their method could be applied to blood or urine samples, but no example was given.

Thus, there exists a need for an electrochemical method to determine oxidative stress by measuring TAC and TOS in a biofluid sample simultaneously, as an alternative to the colorimetric assays.

### The Invention

We have found that TAC and TOS levels could be accurately determined simultaneously in urine samples by voltammetry over an array of working electrodes consisting of one or more bare electrodes and one or more surface-modified electrodes. Such arrays are often named “voltammetric electronic tongues”. Experimental work reported below indicate that chemometric models (a regression model) could be successfully applied to the voltammogram recorded by a suitably designed voltammetric electronic tongue, such that TAC and TOS concentrations predicted by the model fit well to TAC and TOS concentrations excepted on the basis of customary colorimetric assays. The model tested shows good performance, i.e., recovery of ~100%, Pearson correlation coefficient of nearly 1 and very small root mean square error. The correlation was shown between fifteen urine samples of healthy volunteers and TAC and TOS parameters measured by the ABTS and ferrous ion colored complex spectroscopic protocols, respectively.

Voltammetric electronic tongue is a device employing an array consisting of a few working electrodes that are different from one another, i.e., the working electrodes are made of different metals or are surface-modified in a different manner (coated with distinct types of films), as described, for example, by del Valle, M. Electronic tongues employing electrochemical sensors. Electroanalysis vol. 22 1539-1555 (2010). Thus, the sensor array has semi-selective electrodes and cross-response features to “sense” more information. Then, chemometrics models are used, to deal with interference, allowing simultaneously classifying and quantifying multiple analytes. Voltammetric electronic tongues have also been suggested for use in connection with measurements in biofluids [Saidi et al. Voltammetric electronic tongue combined with chemometric techniques for direct identification of creatinine level in human urine. Meas. J. Int. Meas. Confed. 115, 178-184 (2018); Pascual, L. et al. Detection of prostate cancer using a voltammetric electronic tongue. Analyst 141, 4562-4567 (2016), and co-assigned WO 2018/225058.

In the experimental work reported below, the working electrodes incorporated into a voltammetric electronic tongue for determination of oxidative stress, were selected based on their sensitivities towards ascorbic acid (as a model antioxidant for TAC) and hydrogen peroxide (as a model oxidant for TOS).

Accordingly, the invention is primarily directed to a method of determining oxidative stress in a subject, comprising:
obtaining a biofluid sample from the subject;
acquiring an electrochemical signal from the sample with the aid of an array of bare and surface-modified electrodes that is sensitive towards a model antioxidant and a model oxidant;
optionally preprocessing the electrochemical signal, to obtain processed data;
applying trained chemometric model(s) to the processed or raw data, to measure TAC and TOS levels; and
determining a status of oxidative stress in the subject based on the measured TAC, TOS or the TOS:TAC ratio.

The preferred electrochemical technique to measure TAC and TOS in urine samples is voltammetry (application of varied voltage and measurement of current), such as differential pulse voltammetry (DPV).

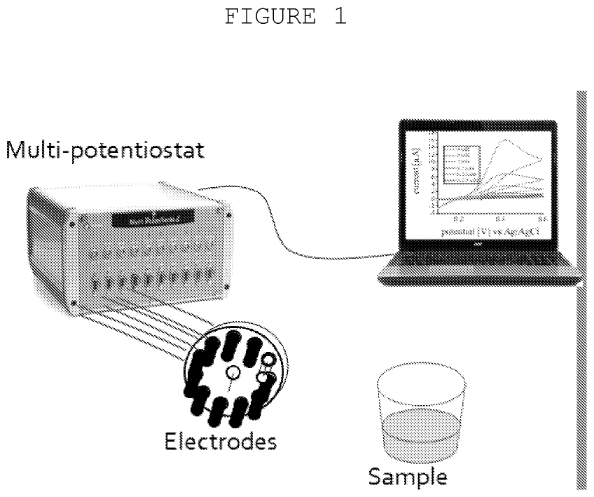

FIG. 1 shows the major components of the approach to urinary detection of oxidative stress, namely, urine sample is taken from the patient; an array of working electrodes is immersed in the sample; the electrodes are connected to a potentiostat, to vary the potential across the electrodes; voltammogram is recorded and analyzed by a suitably trained chemometric model in a computer to extract the TOS, TAC and TOS:TAC ratio from the data.

In its most general form, the array of working electrodes is an assembly of a few sets of electrodes, each set consisting of electrode(s) of the same type. By electrodes of the same type we mean either bare electrode that are made of the same material (e.g., of the same noble metal), or electrodes coated with the same film material. The number of electrodes of type i is marked nᵢ. For example, five types (sets) of working electrodes are listed below (from which at least two sets, or at least three sets, can be chosen to create an array for voltammetry measurements in urine samples):
1) a set consisting of one or more bare electrodes. Bare electrodes are preferably made of noble metals, e.g., gold, platinum, rhodium and iridium. Also, other electrodes, e.g., carbon electrodes (e.g., glassy), can be incorporated into the array of working electrodes. Gold is generally preferred, both for use as bare electrodes and surface-modified electrodes.
2) a set consisting of electrodes coated with polysaccharide (e.g., chitosan) film; typical film thickness is from 1 to 50 μm, e.g., 5 to 20 μm.
3) a set consisting of electrodes coated with polysaccharide (e.g., chitosan) film with conductive additives incorporated into the film, such as carbon nanotubes; typical film thickness is from 1 to 100 μm, e.g., 5 to 60 μm.
4) a set consisting of electrodes coated with reduced graphene oxide film; typical thickness if 200 to 1,000 nm, e.g., 350 to 550 nm.
5) a set consisting of electrodes coated with platinum black film; typical film thickness is from 1 to 50 μm, e.g., 6 to 10 μm.

(coating thickness can be measured by atomic force microscopy or profilometry).

nᵢ—the number of electrodes in each set—is usually up to 3. In fact, because the volume of a biofluid sample such as urine sample may be limited, an efficient array design calls for a small number of working electrodes which could easily fit into a suitable electrochemical measurement cell, i.e., by selecting less than five types of electrodes and reducing the number of electrodes of each type (set).

But the urinary detection of oxidative stress with the aid of a voltammetric electronic tongue is not limited to the use of electrodes coated with platinum black, reduced graphene oxide, chitosan and CNT-added chitosan. The experimental work reported below shows how to apply suitable selection criteria so as to create an effective array with a fairly small number of bare and coated electrodes, with good sensitivity towards ascorbic acid and hydrogen peroxide, such that a chemometric model accurately predicts the total effect of all antioxidants and oxidants present in the urine sample, enabling TAC and TOC determination and accordingly, the oxidative stress index.

Screening tests that can be applied to identify an effective combination I of bare and surface modified electrodes, from a group G of electrodes (I⊆G) are now described, based, as mentioned above, on ascorbic acid as model antioxidant (sometimes abbreviated herein AA) and H₂O₂ (model oxidant); but it is possible to carry out similar screening tests to identify effective electrodes using other TAC and TOC candidate markers instead of ascorbic acid and H₂O₂, such as Trolox and uric acid as alternatives to AA.

First, the electrochemical performance of coated electrodes can be examined by cyclic voltammetry in ferrocyanide/ferricyanide redox couple [Fe(CN)₆³⁻↔Fe(CN)₆⁴⁻] solution—a benchmark frequently used to assess the acceptability of surface-modified electrodes.

Next, cyclic voltammetry can be used to assess the detectability of AA and H₂O₂ by each of the candidate working electrodes in G, by measuring the concentrations of AA and H₂O₂ (each separately) in PBS, across applicable concentration ranges, say, from 0.1 to 10 mM, to show linearity of peak versus concentration curves. The slope of the linear equation fitted to the curve indicates the sensitivity of the tested electrode i towards the analyte (AA or H₂O₂), and is designated S_{i,AA} or S_{i,H2O2}. Working electrodes (bare or surface modified), with sensitivity above 3.0 mA/M, either towards AA (S_{i,AA}>3.0 mA/M) or H₂O₂ (S_{i,H2O2}>3.0 mA/M) are generally preferred.

Partial selectivity is yet another useful selection criterion. Partial selectivity (PS) is calculated by dividing the sensitivity of electrode i towards ascorbic acid by the sensitivity of the same electrode towards H₂O₂. The ratio obtained is PS_{i,AA/H2O2} (or its inverse, PS_{i, H2O2/AA}):

$${PS}_{i , AA / H 2 O2} = \frac{{s}_{i , AA}}{{s}_{i , H 2 O 2}}$$

According to a preferred variant of the invention, the array used for electrochemical urinary detection of oxidative stress in a subject comprises at least one working electrode showing partial selectivity for ascorbic acid/hydrogen peroxide of above 5

$$( that is , \frac{{s}_{i , AA}}{{s}_{i , H 2 O 2}} > 5 )$$

and at least one working electrode showing partial selectivity for hydrogen peroxide/ascorbic acid of above 2

$$( that is , \frac{{s}_{i , H 2 O 2}}{{s}_{i , AA}} > 2 ) .$$

For example, bare gold electrode and platinum black coated electrode show PS_{i,AA/H2O2} above 5 and PS_{i,H2O2/AA} above 2, respectively, and are therefore especially preferred for use in the invention. In fact, the platinum black-coated electrode seems to be quite unique in that it demonstrates increased partial selectivity towards hydrogen peroxide compared to the other electrodes.

Cross reactivity can then be calculated over a combination I consisting of, say, three different types of electrodes (I⊆G):

$${CR}_{I , j} = \frac{1}{2} {\sum }_{i ∈ I} {\sum }_{k ∈ I} \vert {PS}_{i , j} - {PS}_{k , j} \vert$$

where i and k are two different types of electrodes in the combination I and j is the analyte. Combination I can be then be rejected or accepted based on its CR. For example, for G={bare electrode, chitosan-coated electrode, chitosan+CNT coated electrode, Pt coated electrode and rGO coated electrode} useful three-electrode combinations I determined by the CR test were:
I1={bare electrode, Pt coated electrode, chitosan+CNT coated electrode};
I2={bare electrode, Pt coated electrode, chitosan coated electrode};
I3={bare electrode, Pt coated electrode, rGO coated electrode}.

The coatings mentioned above (made of platinum black, reduced graphene oxide, chitosan and chitosan to which conductive additive was added, such as carbon nanotubes) are applied onto surfaces of bare electrodes (e.g., on 2 to 3 mm disc shape commercial gold electrode) by a suitable electrodeposition technique:
(i) galvanostatic electrodeposition (chronopotentiometry), in which a constant current is passed through the electrode(s) to be coated;
(ii) potentiostatic electrodeposition (chronoamperometry), in which a constant potential is applied on the working microelectrode(s) to be coated; or
(iii) cyclic voltammetry electrodeposition.

Platinum black film can be generated via galvanostatic electrodeposition onto one or more electrodes, by passing a constant current (a cathodic current, with current density fixed in the range of 0.1 to 4 mA/cm², for 3 to 7 minutes; for example, a current density of 0.3 mA/cm² is supplied over five minutes), through a deposition solution in which a suitable platinum source is dissolved, e.g., by electrochemical reduction of chloroplatinic acid dissolved in DI water at a concentration in the range of 1% (v/v in water) to 3% (v/v in water), in the presence of about 0.05% (v/v in water) of lead acetate. Lead acetate enhances the electrode reaction (i.e. the reduction of Pt) in presence of platinum black solution and it also strengthens the adhesion of the coating to the electrode. The pH of the deposition solution is shifted to the strongly acidic by addition of hydrochloric acid. A two-electrode configuration can be used, which includes the electrodes to be coated as working electrode(s) and a ring or wire Pt counter electrode.

Another type of film-forming material that is applied to create film-coated microelectrode(s) is reduced graphene oxide. The deposition solution is prepared by known methods, e.g., the Hummers' method, where oxidation of graphite flakes or powder takes place upon adding the graphite to a cold solution of sulfuric acid (e.g., 0 °C) followed by gradual addition of sodium nitrate and potassium permanganate under continuous stirring. For example, on a laboratory scale, the addition time of each of the successively added NaNO₃ and KMNO₄ reagents is not less than ten to fifteen minutes. On completion of reagent's addition, the reaction mixture is heated to about 35-45 °C and kept under stirring for a couple of hours, e.g., not less than two hours. The reaction is terminated by addition of water and hydrogen peroxide which removes excess permanganate. The graphene oxide (GO) is recovered by centrifugation and freeze dried and used to prepare deposition solution with concentrations in the range from 0.1 to 0.9 mg/ml GO. A deposition solution can also be prepared by a modified Hammers procedure, which consists of adding the graphite powder (or flakes) to a mixed sulfuric acid/phosphoric acid solution (e.g., proportioned about 9:1 by volume), followed by the slow addition of KMnO₄. The mixture is kept under stirring for couple of hours at a slightly elevated temperature (at 30-35 °C) until the mixture acquires a dark green color. Termination of the reaction is achieved by slow addition of H₂O₂ aqueous solution (e.g., the commercial 30% w/w solution). Graphene oxide is recovered through acidification of the mixture by hydrochloric acid (e.g., addition of commercial 32% HCl solution and DI), centrifugation of the resulting solution, washing of the supernatant with HCl/water, drying of the washed solution (e.g., at 90 °C in an oven) and collecting the GO powder. The dried GO powder is dissolved in DI, usually up to concentration of 0.5 g/L GO concentration. Addition of an electrolyte to the GO solution affords the GO electrodeposition solution. Next, r-GO is obtained electrochemically from the GO solution onto the electrode (e.g., Au) surface, using cyclic voltammetry electrodeposition, in a three-electrode cell configuration consisting of the microelectrode(s) as working electrode(s); an externally applied Pt wire as counter electrode and Ag/AgCl as reference electrode. The GO solution is added to the chamber; a potential window, for example from −1.4. to 1.4V (versus Ag/AgCl) is scanned at rate of in the range of to 50 to 500 mV/s, with number of cycles varying from 1 to 5.

Electrodeposited chitosan film-coated microelectrode can be prepared with the aid of a deposition solution with chitosan concentration in the range from 0.5 to 2.0 wt %, preferably from 0.8 to 1.2 wt %, prepared by dissolving chitosan in a strongly acidic environment, whereby the amino groups undergo protonation to reach a slightly acidic pH (5-6). As pointed out above, conductive additives can be included in the deposition solution; these additives will co-deposit and affect the film properties. The concentration of the additives in the deposition solution (e.g., carbon nanotubes (abbreviated herein CNT), gold nanoparticles and platinum nanoparticles) is in the range from 0.1 to 2%, preferably from 0.8 to 1.8 wt %. For example, chitosan-CNT electrodeposition solution can be prepared by mixing a chitosan solution as previously described with CNTs, followed by ultra-sonication. The electrode is immersed in the chitosan deposition solution (or chitosan/CNT solution) and electrodeposition is achieved by the chronopotentiometry technique, i.e., selected electrodes to be coated are biased to the negative potential against a counter electrode with constant (cathodic) current being applied between the electrodes for a period of time of 0.5 to 5 min, supplied by a DC current source; typically the current is set in the range from 3 to 6 μA/cm². A two-electrode configuration can be used, i.e., the counter electrode is shorted to reference terminal. Weakly bound chitosan is removed from the microelectrode surface, by immersing the device in a buffer solution.

The arrays of working electrodes, for example:
n_{bare}=n_{chitosan}=n_{chitosan+CNT}=n_{Pt}=n_{rGO}=2 (a total of ten electrodes);
n_{bare}=n_{chitosan+CNT}=n_{Pt}=2 (a total of six electrodes) may be arranged in different designs and geometries.

For example, one simple and straightforward configuration is based on the use of an electrochemical measurement cup to hold the urine sample (e.g., not less than 10 ml sample), fitted with a perforated cover; the holes in the cover correspond in number and size to the electrodes, such that individual working electrodes can be inserted into the measurement cell through the holes to be immersed in the sample. Commercial counter electrode (e.g., commercial Pt wire) and commercial reference electrode (Ag/AgCl) are also inserted into the cup. Such a design was used in the experimental work conducted in support of this invention and is described in more detail below.

A more sophisticated design is the one shown in WO 2018/225058. It was based on a cylindrical body made of silicon, polyvinyl alcohol or polydimethylsiloxane, which was 2 to 5 cm long and with diameter is in the range from 2 to 3 cm. The accessible surfaces of the electrodes were deployed on one base of the tubular body: a disc-shaped reference electrode positioned concentrically and coaxially in respect to the cylindrical body, a ring-shaped counter electrode encircling the reference electrode (ring area of at least 5 mm²) and multiple surface modified working electrodes (1 mm² each) positioned in radial direction from the reference and counter electrodes and evenly distributed along the perimeter of the base of the cylindrical body. The opposite base provides the electrical wiring to be connected to potentiostat/galvanostat (the electrodes extend along the cylindrical body and are connected to the wiring in the opposite base). When put to use, the electrochemical sensor is immersed in the urine sample to be analyzed such that the base of the cylinder, where the electrodes are disposed, is exposed to the sample allowing the electrodes that (optionally) protrude from the base to be dipped into the urine sample, creating the electrochemical cell for the measurements.

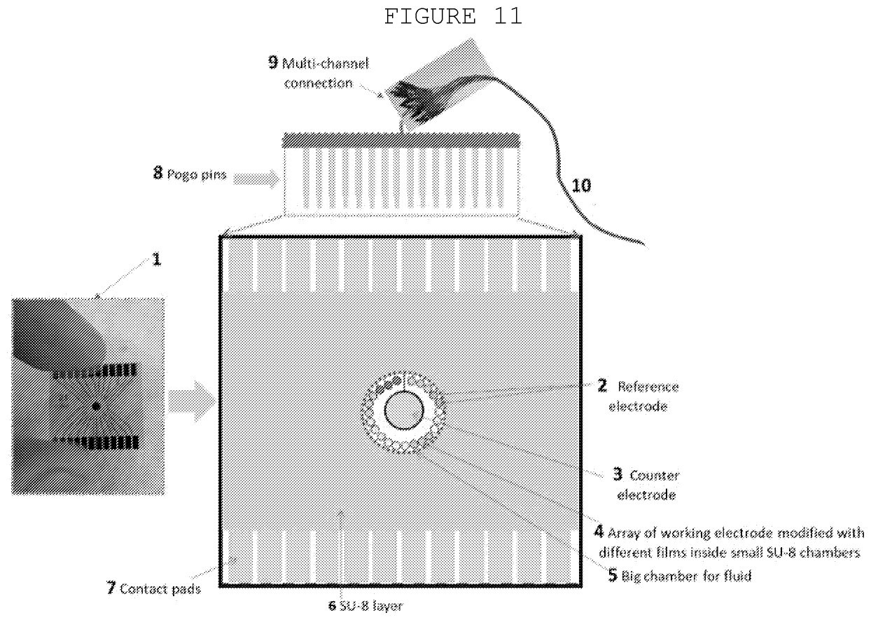

Alternative designs based on microfabricated configurations can also be considered for the electrochemical sensor. One example is shown in FIG. 11. An electrochemical sensor in the form of a microfabricated 1.5 cm×1.5 cm chip (1) on a glass substrate is shown. It can be a portable device or can be placed in the lab. The device dimensions are compatible with the conventional microfabrication techniques where the diameter of the working microelectrodes (4) are ~100 micrometer and the diameter of counter electrode (3) is ~500 micrometer. The chamber (5) is designed to hold small volume samples (10-30 microliter). Reference electrode (2) can be integrated into the array by electroplating one or two microelectrodes with Ag/AgCl as previously described (e.g., WO 2022/137236). There are two kinds of chambers, a small chamber for each microelectrode opening (4 and 3) and a bigger chamber to carry the fluid sample (5). The chambers are made of insulating polymer, e.g., SU-8 polymer (6). The contacts pads (7) can be connected via pogo pins (8) and then to the multichannel connection (9) of the potentiostat or galvanostat unit (10; not shown). The device may be powered by a battery or alternatively, can be connected to a main power supply. A control unit (not shown) is designed to serve several purposes, chiefly controlling the potential of the working electrodes or the current flowing through the cell, respectively, according to the chosen electrochemical technique.

The microsensor described above (which consists of microelectrodes, microchambers encompassing the microelectrodes, all confined within a recessed zone that serves as a receptable for holding the liquid sample) can be created over a substrate by techniques such as etching and photolithography. Briefly, a substrate is cleaned, a first photoresist is applied (either negative, positive or image reversal resist), e.g., by spin coating, spray coating or dip coating, to produce a thin uniform layer on the substrate, followed by soft baking. A first mask is aligned, to transfer the pattern corresponding to electrodes' sites onto the surface of the substrate. The photoresist is exposed through the pattern on the mask with UV light, followed by a development step. Next, bare microelectrodes are deposited in the intended sites, e.g., first titanium which serves as an adhesion layer and then gold followed by lift off procedure that resulted in a gold microelectrode array on glass substrate. In order to define the electrode effective surface area, another lithography step was done using, e.g., SU-8 photoresist. To define the chamber for fluid, another photolithography step was followed with e.g., thick SU-8 resist. See WO 2022/137236 for a complete protocol.

Having patterned the microstructures on the substrate, the desired coatings are applied on the gold microelectrodes, for example, by electrodeposition.

In operation, the electrodes are electrically connected to potentiostat or galvanostat which control the potential or current of the working electrodes, respectively, to create a data set of electrochemical signals when the electrodes are in contact with the test biofluid (e.g., urine) sample. The data set of electrochemical signals is analyzed by a processor applying one or more chemometric techniques.

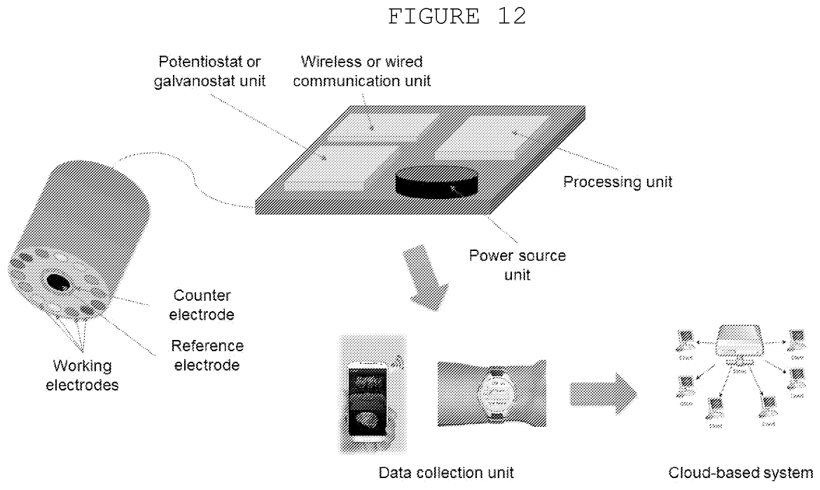

FIG. 12 provides a schematic illustration of the electrochemical sensor according to the invention and a detection device into which the sensor is incorporated, i.e., either a portable device or a fixed device placed in a lab etc. The device may further include a data storage unit or a data transmitting unit, i.e., wired transmitter or a wireless network transmitting unit with conventional communication ports to deliver the data to an externally located data storage unit.

A data storage unit may be the memory of the data processing unit or any computer readable media. In FIG. 12, personal instruments are shown and also a cloud-based data storage system.

The general structure of such devices is described in WO 2022/137236. The device further comprises a processor for analyzing a data set of electrochemical signals by one or more chemometric techniques, e.g., multivariate methods such as a supervised machine learning model (artificial neural network (ANN)), or a regression model, e.g. partial least square regression (PLSR).

Briefly, PLSR is a linear regression method and PLSR algorithms are available (e.g., MATLAB). As to ANN, a neural network model is generated with the aid of a training set. To this end, a matrix consisting of large number of samples with known concentrations of the analyte and with known outputs is collected. As explained in more detail below, the data set is split to create a training set, a cross-validation set and a test set. In the training process, the error between the outputs predicted by the neural network and the known outputs is calculated; this process continues, with the algorithm adjusting the parameters iteratively to minimize the error, i.e., to reduce the error below an acceptable level. Once created, the model is saved and can be used for future measurements of test samples.

It should be noted that raw test data collected by the electrochemical sensor undergoes pre-processing with the aid of known techniques before it is fed to the algorithm. Then methods such as principal component analysis (PCA), Fast Fourier Transform (FFT), and selection of important electrochemical signal features, can be used to reduce the dimensions of the data fed to the model. The latter method has been shown to be especially useful; the features selected (e.g., from the voltammograms) include peak current, peak potential, maximum slopes of the I vs. E function (for the increasing and decreasing parts of the function).

That is, to make a measurement of a test sample—using voltammetry for example—the sample is placed in the sample holder in contact with the electrochemical sensor in the device of the invention, as described above, varied voltage is applied by the potentiostat between the reference electrode and working electrodes, currents generated are measured and the measurements are stored, and the test data collected (readings from all working electrodes) is preprocessed, reduced and scaled, fed to the ANN algorithm to obtain the model input.

The two approaches for model building—PLSR and ANN are now discussed in more detail; the major steps are outlined below. In both cases, data reduction is based on signal features.

### Model Building Process—Based Signal Samples (PLSR)

1. Organization of data in a cell structure—with the aid of MATLAB software reading csv files, all experimental data is arranged in one type of structure (e.g. cell type).
2. Signal smoothing—by using the signal processing toolbox, MATLAB software 2017a version, a built-in function (e.g. ‘filter’) can be used to filter the signals by employing a moving average window in order to reduce signal fluctuations and noisy behavior which is not originated by the electrochemical properties of the tested solution. A varied filter order in the range of 5<M<8, (M—filter order), depending on the noise level in the recorded data, can be used. In order to keep this parameter as unbiased for all the recorded signals in each experiment, it is kept fixed and equal to specific value for each experimental data.
3. Baseline subtraction—in an electrochemical analysis, the main interest is the faradaic current that is generated owing to the electron transfer from the redox molecule to the electrode surface in a specific electric potential (oxidation potential). In order to improve signal to noise ratio (SNR), the Asymmetric least squares spline regression (AsLSSR) was used. With the aid of MATLAB software 2017a version, a function is built to estimate the baseline signal by getting two constant values parameters, λ the smoothing parameter (10²<λ<10⁹) and p the asymmetry parameter (0.001<p<0.1). These two parameters take part in the numerical optimization of the cost function of the algorithm.
4. Organization of signals in a matrix structure—the signals are arranged in a matrix form, with each raw corresponding to specific array response. Signals were put in the matrix one after the other, to produce a super raw vector structure for each solution, while the target was defined as the concentration matrix, each column describing specific analyte concentration used through the experiments. This has been achieved by building MATLAB script (version 2017).
5. Dividing the data set into distinct subsets—the data is separated into two or three distinct sets. The first set is a training set, that is used for the training and the design of the model. All optimization procedures for finding the optimal solution are performed on the training set. It should be noted that the training set could be sub-divided to create a small cross-validation set. The other set is the test set. This set is used to check the model's generalization capabilities, by using the trained model in order to evaluate ability of the model to predict the concentrations in the “unseen” samples. The data is usually divided as follows: 70-85% of the samples are assigned to the training set (including ~10% that may be used for cross-validation) and 10-30% for testing. The samples are divided randomly, but the computer's random generation is fixed to assure that the same subdivision could be reproduced.
6. Signals centering—in order to focus on the variability of each specific potential, data is centered, checking the average features value for the all set, and subtract it from the all signal, resulted with features with mean value equal to 0. The average value of the training set is saved for future use for centering the test set.
7. Choosing a regression model for prediction analysis—the partial least square regression (PLSR) model, a linear technique, was used. It is especially suitable for cases where there is a high correlation between the different features and when there is a limited number of samples (e.g. solutions). The ‘plsregress’ MATLAB function toolbox was used for model building and testing.
8. Choose optimal model parameters (k-fold cross validation)—In order to choose wisely different digital (e.g. number of latent variable in a PLSR model) and physical parameters (e.g. electrode combination) the CV method (LOOCV and 10-fold CV) was used. With the aid of a code that is able to give all the possible configurations without repetition, the CV was implemented in the MATLAB software 2017a version, using the ‘cvpartition’ function from the statistical toolbox, for random divisions into k sets. By dividing the train set and using it also for validation we were able to take advantage of most of the information hidden in the data. Model parameters minimizing the cross-validation error were chosen.
9. Model training—the best number of latent variables and best electrode combinations were chosen for training the model on all the training set. A PLSR model using the ‘plsregeress’ function from MATLAB statistics tool box (2017 version) was built.
10. Test Data pre-processing—the test signals were centered according to the mean average value of the training set.
11. Model predictability—the trained model was used to test and evaluate the performance on unseen data set, i.e., the test set, which was preprocessed and was ready for use as the model input.
12. Evaluate model performance—the quality of the model is assessed with the root mean square error between the known concentrations and those that were estimated by the model.

$${RMSE}_{test} = \sqrt{\frac{1}{{N}_{test}} {\sum }_{i = 1}^{{N}_{test}} {( {C}_{expected} - {C}_{calculated} )}^{2}}$$

(N is the number of samples; C_{expected} is the real actual value and C_{calculated} is the predicted value).

### Model Building Process—Based Direct Electrochemical Features (ANN)

1. organization of data in a cell structure—with the aid of MATLAB software, csv files are read, in order to arrange all the experimental data in one type of structure (e.g. cell type).
2. Signal smoothing—by using the signal processing toolbox, MATLAB software 2017a version, a built-in function (e.g. ‘filter’) is used to filter the signals by employing a moving average window in order to reduce signal fluctuations and noisy behavior which is not originated by the electrochemical properties of the tested solution. A varied filter order in the range of 5<M<8, (M—filter order), depending on the noise level in the recorded data, is used. In order to keep this parameter as unbiased for all the recorded signals in each experiment, it was kept fixed and equal to specific value for each experimental data.
3. Feature extraction—specific electrochemical signal features were extracted, i.e., features which are indicative of the identity of the redox-active molecule and its concentration in the solution. The extracted features include: peak potential, peak current, maximum slope of the signal, and current value at specific potentials (potentials which are known as the standard oxidation-reduction potential of specific analyte—good evaluation when the peak is not visible). All features extracted automatically using MATLAB software 2017a version built-in functions and by customary-built specific functions for each feature.
4. Organize features in a matrix structure—the extracted features were arranged in a matrix form, with each raw corresponding to specific array response, whereas each column describes specific analyte concentration through the experiment. This has been done by building MATLAB script (version 2017).
5. Dividing the data set into distinct subsets—The data is separated into two or three distinct sets. The first set is a training set, that is used for the training and the design of the model. All optimization procedures for finding the optimal solution are performed on the training set. It should be noted that the training set could be sub-divided to create a small cross-validation set, as explained below and further illustrated in the Examples below. The other set is the test set. This set is used to check the model's generalization capabilities, by using the trained model in order to evaluate ability of the model to predict the concentrations in the “unseen” samples. The data is usually divided as follows: 70-85% of the samples are assigned to the training set (including ~10% that may be used for cross-validation) and 10-30% for testing. The samples are divided randomly, but the computer's random generation is fixed to assure that the same subdivision could be reproduced.
6. Feature normalization—features were standardized using the z-score transformation (subtracting the mean value of each feature, and scaling it by dividing the value by the standard deviation). Scaling was preformed because the features were in different scales, such as peak currents [μA] and peak potentials [V]. The data transformation was achieved with the aid of MATLAB software 2017a version. The transformation was performed on the training set, when the moments values were saved for future scaling of the test data.
7. Feature selection—The strategy employed for data reduction to decrease computational complexity was ten-fold cross-validation forward selection based linear regression. The criterion for the selection was the root mean square error between the “real” concentration and those estimated for the validation set. This was achieved with the aid of the statistical toolbox of MATLAB software 2017a version. In each the experiments we used a different initial number of features depending on the technique that was chosen to extract data features.
8. Choosing model for prediction analysis—In order to perform multivariate analysis (not only one target value), artificial neural network (ANN) models were used—a nonlinear techniques—to explore the relation between the extracted features to the analytical properties (such as total antioxidant capacity (TAC), total oxidant status (TOS), a specific reactive oxygen species concentration (e.g., H₂O₂), and a specific antioxidant species concentration (e.g., ascorbic acid)). The ANN MATLAB toolbox was used to explore different network architectures.
9. ANN model optimization (based k-fold cross-validation)—Simple ANN architectures, such as 1-hidden layer with limited number of neurons, was used in order to reduce the chance for overfitting—the lesser number of neurons in use the lower network complexity. The best architecture was chosen with the aid of a cross-validation test: the number of neurons in the hidden layer was varied to test the network performance on a validation set. The upper bound of the number of neuros was set such that it is smaller than the number of the model weights. Then the number of neurons with the best score (in terms of the root mean square error between the known concentration and those who were estimated on the validation set) was chosen. The test was repeated with different initial conditions (e.g. different weight initializations), because ANN models are significantly affected by their initial conditions; but in each individual test the parameters were fixed in order to make unbiased and robust decision
10. Model training—having determined the best architecture, it was now used for training the model across the entire training set. The number of the training iterations was limited (early stopping) according to a specific error value that was set to stop the training procedure after reaching at least 99% of the target variance. Hence a trained network which minimizes the performance on the training data is created, ready for future testing.
11. Test data pre-processing—based on the selected features in the feature selection procedure, the test features were loaded and standardized according to the training moments. For each feature, the training mean value was subtracted and the result divided it by the training standard deviation (this procedure is based on the fact that the two sets sampled from the same data population), creating a scaled data set.
12. ANN predictability—The trained model was used to test and evaluate the performance on unseen data set, i.e., on the test set which was preprocessed and was ready for use as the model input. Calculations were performed in MATLAB software 2017a version, using the ANN toolbox function and aid function coded for specific tasks.
13. Evaluation of model performance—the quality of the model is assessed with the root mean square error (between the known

$${RMSE}_{test} = \sqrt{\frac{1}{{N}_{test}} {\sum }_{i = 1}^{{N}_{test}} {( {C}_{expected} - {C}_{calculated} )}^{2}}$$

(as previously defined) and the Pearson correlation coefficient (PCC):

$$PCC = \frac{E [ {C}_{expected} - {μ}_{expected} ] E [ {C}_{estimated} - {μ}_{estimated} ]}{{σ}_{expected}^{2} {σ}_{estimated}^{2}}$$

The composition of the electrodes in the array can be chosen based on various options: 1) high partial selectivity (PS) versus the analyte in the presence of interfering molecules, 2) high cross reactivity (CR) scores, and 3) high prediction score based on the tested chemometric model (PRESS in the case of PLSR).

The features extracted from the electrochemical signal can be used as is from the raw data (such as currents at specific applied potentials) or following data processing (such as dimension reduction to several PCs by using PCA).

The chemometric model to predict TAC and TOS levels in real clinical samples can be trained with simulated solutions (such as simulated urine) spiked with various levels of the analytes or with clinical samples (such as urine samples from volunteers).

### EXAMPLES

### Preparations 1-4

### Electrodeposition Solutions

### 1) Chitosan Electrodeposition Solution (1 wt. %)

9 g chitosan powder was dissolved in 500 ml DDW and stirred for 20 min. 10 ml 2M HCl solution was added to the solution to reach pH of 5.5. The solution was sonicated for 45 minutes and then stirred again for 90 minutes at 700 rpm. The solution was filtrated with a metallic mesh 0.2 mm cylinder.

### 2) Chitosan-Carbon Nanotube Electrodeposition Solution (1 wt. %)

200 mg carbon nanotube, multi walled, was added to 20 ml of the 1 wt. % chitosan solution and stirred for 15 minutes in 250-500 rpm. Then the solution was sonicated for one hour. The solution was used during a storage period of one week.

### 3) Platinum-Black Electrodeposition Solution

Platinum black deposition solution was prepared by mixing 0.5 g of chloroplatinic acid and 25 mg of lead acetate in 50 ml of DI water. The mixture was then stirred and 3.9 μL of concentrated hydrochloric acid (32%; 10.2 Molar concentration) was added to the solution. The prepared solution was covered with aluminum foil and stored at room temperature.

### 4) Graphene Oxide Electrodeposition Solution

10 ml of 1 mg/mL rGO electrodeposition solution was prepared by diluting 2.5 ml of graphene oxide 4 mg/mL solution, with NaCl 100 mM in 5.5 ml DDW. The graphene oxide (GO) solution was prepared using a modified Hummers' method. A 9:1 ratio of sulfuric acid and phosphoric acid (100 mL) was prepared and stirred for several minutes. A graphite powder (7.5 g/L, 1 wt. eq.) was added to the mixture under stirring conditions. Potassium permanganate (45 g/L, 6 wt. eq.) was slowly added to the solution and the mixture was stirred for 6 h at 30-35 °C until the color turned to dark green. To eliminate the excess of potassium permanganate, hydrogen peroxide 30% w/w (2.5 mL) was added slowly and the mixture was stirred for 10 min, resulting in an exothermic reaction that was left to cool at room temperature. Concentrated 32% hydrochloric acid and DI were sequentially added at a 1:3 volume ratio and the resulting solution was centrifuged at 7000 RPM for 5 min. Residuals of the centrifuged solution were washed 3 times with hydrochloric acid and DI (1:3 v/v). The washed GO solution was dried at 90 °C in an oven (Binder—9010-0082) overnight, yielding the GO powder.

### Preparation 5

### Fabrication of the Electrochemical Biosensor

### Surface Modification of Electrodes

2 mm diameter commercial gold electrodes were used (CH Instruments). Prior to coating, the surface of the electrode was polished by using a series of 1.0, 0.3, and 0.05 μm α-Al2O3 slurry on a micro-cloth pad until a mirror-shiny surface was obtained. The polished electrode was further rinsed with double-distilled water. The electrochemical activity of the polished electrode was validated after each polishing procedure by cyclic voltammetry (CV) measurement in 5 mM ferrocyanide/ferricyanide (ferro/ferri) solution to get signal as clean commercial electrode (0.037 mA). For validation by CV the next parameters were used: potential range −0.1V to 0.57V, scan rate 50 mV/sec. Then each electrode was rinsed with double distilled water (DDW) and coated with appropriate coating.

The following coating were electrodeposited onto the gold electrodes with VSP-300 Biologic potentiostat and EC-Lab software:

### Chitosan-Coated Electrodes

A chronopotentiometry technique was employed to electrodeposit chitosan from the solution of Preparation 1 onto gold electrodes over 300 s, at cathodic current density of 6 A/m², using a two-electrode configuration (a platinum wire as a counter electrode, and the gold as a working electrode). The modified electrodes were rinsed in double-distilled water (milli-Q, 18 MΩ) to remove chitosan that was not bound to the electrode.

### Chitosan-Carbon Nanotubes Coated Electrodes

A protocol akin to the one described for the chitosan electrodeposition was used to form chitosan-CNT coatings on gold electrodes (see Preparation 2 for the electrodeposition solution).

### Platinum-Black Coated Electrodes

A protocol akin to the one described for the chitosan electrodeposition was applied, but this time a cathodic current density of 0.3 mA/mm² was passed through the electrodeposition solution.

### Reduced Graphene Oxide Coated Electrodes

Cyclic voltammetry (CV) technique was employed for the electrodeposition, cycling 5 times across the potential range of −1.4 V to 1.4 V (vs. Ag/AgCl), at a scan rate of 0.05 V/s. A three-electrode cell configuration consisting of the gold electrode (working electrode; ‘WE’), an externally applied commercial Pt wire (CHI115, CH Instruments; counter electrode; ‘CE’), and a Tungsten needle (P/N H-20242, Quarter) coated with Ag/AgCl ink (011464, BAS Inc.; pseudo reference electrode; ‘RE’).

### Assembling Electrode Array

The array design consists of ten working electrodes divided equally into five sets as follows:

n_{bare}=n_{chitosan}=n_{chitosan+CNT}=n_{Pt}=n_{rGO}=2

The geometrical arrangement of the electrodes is illustrated in the experimental setup that is shown in the next set of Examples. The electrodes were inserted into the solution/sample in an electrochemical cell through the holes of a suitably perforated cell cap; the set of working electrodes and one reference electrode were arranged along the circumference of a circle encircling a centrically positioned counter electrode.

### Example 1

### Response Validation

### Part A: Spectrophotometric Response Validation

### Reagents and Solutions

TAC assay based on the ABTS (2,2′-Azino-bis(3-ethylbenzothiazoline-6-sulfonic acid) test: the first reagent is 0.4M acetate buffer solution (pH 5.8) that contains sodium acetate solution (470 ml) and acetic acid solution (30 ml). The acetate solution contains 16.4 g sodium acetate dissolved in 500 mL of ultrapure water. The acetic acid solution contains 1.1484 ml of reagent-grade glacial acetic acid that was added to 48.8516 ml ultrapure water. The second regent is ABTS radical in acetate buffer (30 mM, pH 3.6). The acetate buffer solution contains 37.5 mL sodium acetate solution that was mixed with 462 mL of the acetic acid solution. The sodium acetate solution contains 1.23 g sodium acetate dissolved in 500 mL of ultrapure water. The acetic acid solution contains 861.3 μL of reagent-grade glacial acetic acid was added to 499.1387 mL of ultrapure water. Then, 8.0833 μL of H₂O₂ was diluted in 25 ml acetate buffer (described above). Then 0.1372 g of ABTS was dissolved in the prepared solution.

TOS assay based on the Fe³⁺ ion and xylenol orange: the first reagent contains 150 μM xylenol orange, 140 mM NaCl, and 1.35 M glycerol. A 250 ml solution of the reagent was prepared from 0.0285 g of xylenol orange, 2.045 g of NaCl, 225 mL of 25 mM H₂SO₄ and 25 mL of glycerol. The second reagent contains 5 mM Ferrous ammonium sulfate, and 10 mM o-dianisidine dihydrochloride. For preparation of 250 mL solution of the second reagent, 0.49 g of (NH₄)₂Fe(SO₄)₂·6H₂O and 0.6107 g of o-Dianisidine dihydrochloride were dissolved in 250 mL of 25 mM H₂SO₄. 25 mM H₂SO₄ was obtained by diluting 25 mL of 0.5 M H₂SO₄ in 475 mL of ultrapure water. The concentrated 0.5 M H₂SO₄ solution was obtained by adding 679.3 μL of H₂SO₄ 18.2M to 24.321 mL DI Water. The solutions were stored for maximum 6 months at 4 °C.

Ascorbic acid solutions: Ascorbic acid solution was prepared at concentration of 4 mM. Then, it was serially diluted in ultrapure water and phosphate buffer saline (PBS) across the range of 0.125 mM up to 4 mM for the calibration curve performance of the spectrophotometric TAC assay and the electrochemical response validation.

H₂O₂ solutions: Two hydrogen peroxide solutions were prepared at concentrations of 4 mM and 400 μM. Then, each of these solutions was serially diluted in ultrapure water and PBS over the range of 0.125 mM up to 4 mM and 12.5 μM up to 400 μM for the calibration curve performance of the spectrophotometric TOS assay and the electrochemical response validation.

The PBS (phosphate buffer saline) was prepared by dissolving 1779.9 mg sodium phosphate dibasic, 244.96 mg sodium phosphate monobasic, 8.006 g sodium chloride and 201.28 mg potassium chloride in 1 L of deuterium-depleted water (DDW), to obtain for 10 mmol/L solution at pH 7.4.

### Spectrophotometric Measurements

Determination of TAC: a 12 μL of a sample and 250 μL of reagent 1 were added to a 96-well plate and incubated for 2 minutes. Then, the absorbance was measured at 600 nm. After that, 25 μL of reagent 2 was added to the reaction. After 5 minutes incubation, the second measurement taken. The difference between the first and second measurements was used to calculate TAC in all assays. The results were expressed in millimoles of ascorbic acid equivalents per liter.

The TOS method based on the reaction that the ferric ion makes a colored complex with xylenol orange in an acidic medium. For TOS assay, briefly, first, 35 μL of sample and 225 μL of reagent 1 were added to a 96-well plate and incubated for 2 minutes. Then, the absorbance was measured at 560 nm followed by secondary measurement at 800 nm. After that, 11 μL of reagent 2 was added to the reaction. After 5 minutes incubation the third and fourth measurements were taken in the same wavelengths as previously. The difference between the first and second measurements, and between the third and fourth measurements were used to calculate TOS in all assays. The assays results were expressed in micromoles of hydrogen peroxide equivalents per liter.

### Results

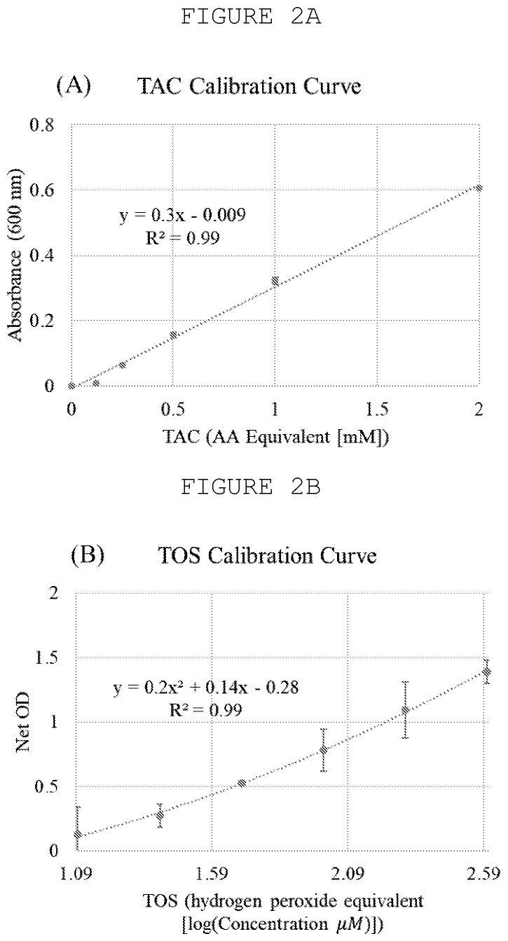

FIG. 2A shows that linear relationship was observed between the AA concentration and the absorbance at 600 nm, as expected. FIG. 2B shows that polynomial relationship was observed between the log(concentration) to the net optical density (OD). in the literature, exponential relationship was observed, nevertheless, polynomial curve led to a better score in terms of R-squared values.

After that, the measurements of the urine samples were fitted according to the calibration curves as a gold standard for the electrochemical sensor for OS measurements development.

Part B: Electrochemical Response Validation of the Ten-Electrode Array Design (n_{bare}=n_{chitosan}=n_{chitosan+CNT}=n_{Pt}=n r_{GO}=2)

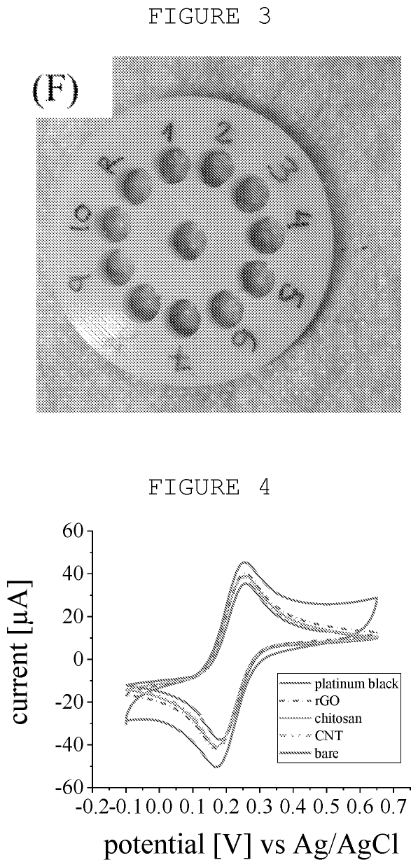

The experimental setup used for the measurements consisted of a 20 ml electrochemical cell that was fitted with a 3D printed cell cap. The cap is shown in FIG. 3. The cap is perforated with twelve holes, which include a centrically located hole for positioning Pt counter electrode and eleven holes arranged in a ring fashion encircling the Pt counter electrode, to position the ten working electrodes (their holes are labeled by numbers 1 to 10) and Ag/AgCl reference electrode (its hole is labeled by the letter R). The electrodes inserted into the solution through the holes were connected to a potentiostat and a computer (Ivium potentiostat and IviumSoft software).

The experimental setup was used to characterize the electrochemical performance of the coated electrodes towards the [Fe(CN)₆³⁻↔Fe(CN)₆⁴⁻] benchmark (part B1) and markers associated with TAC (ascorbic acid) and TOS (hydrogen peroxide) (part B2).

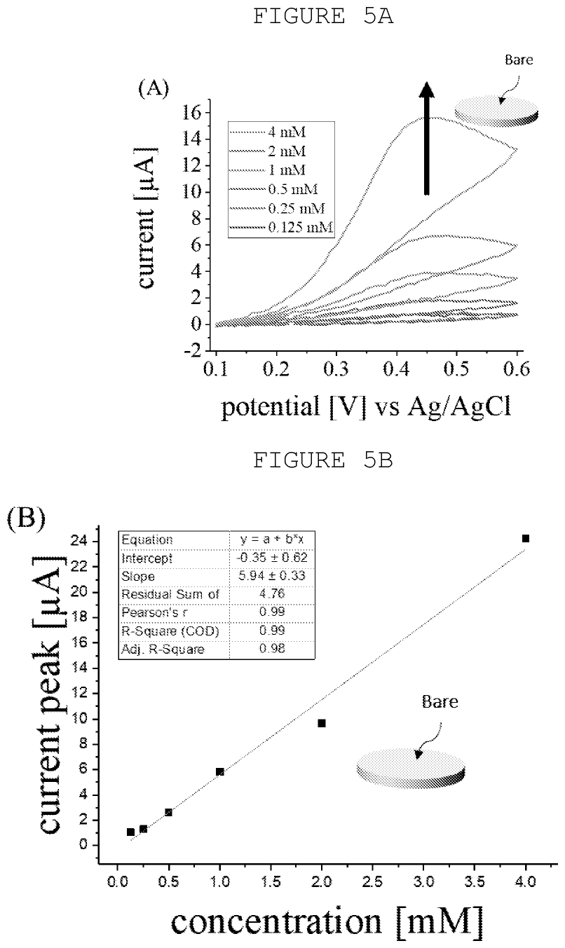

B1: Cyclovoltammetry in a 5 mM solution of the ferrocyanide/ferricyanide redox couple [Fe(CN)₆³⁻↔Fe(CN)₆⁴⁻] was performed across the potential range of −0.1V to +0.65V, for a total of three cycles. The voltammogram is shown in FIG. 4, indicating the detectability of the [Fe(CN)₆³⁻↔Fe(CN)₆⁴⁻] redox reaction by the bare and surface-modified electrodes. Next, diluted solutions with concentrations spanning the range from 4 mM to 0.125 mM (dilution by factor ½) were prepared to create calibration curves for each type of electrode towards the [Fe(CN)₆³⁻↔Fe(CN)₆⁴⁻] analyte, to determine LOD of sensitivity of each electrode. LOD of electrode i {i=bare, chitosan, chitosan+CNT, Pt and rGO} towards analyte j {j=[Fe(CN)₆³⁻↔Fe(CN)₆⁴⁻]} was calculated by the formula:

$${LOD}_{i , j} = 3 \times \frac{PBS std}{{s}_{i , j}} \qquad ( 1 )$$

where PBS std is the standard deviation of the current peak of the analyte taken from 50 PBS samples and S_{i,j} denotes the sensitivity, which is the slope of the current versus concentration calibration curve, of electrode i toward analyte j. The results are tabulated in Table 1.

TABLE 1

| Electrode | Sensitivity [mA/M] | LOD [mM] |
|---|---|---|
| bare | 6.96 ± 0.15 | 0.021 ± 4.4 · 10⁻⁴ |
| chitosan | 5.84 ± 0.8 | 0.026 ± 3.56 · 10⁻³ |
| Chitosan + CNT | 9.71 ± 0.9 | 0.017 ± 1.63 · 10⁻³ |
| platinum black | 8.14 ± 0.9 | 0.022 ± 2.47 · 10⁻³ |
| rGO | 11.1 ± 0.38 | 0.015 ± 5.07 · 10⁻⁴ |

It is seen that the surface-modified electrodes, with the exception of the chitosan-coated electrode, show enhanced selectivity towards the [Fe(CN)₆³⁻↔Fe(CN)₆⁴⁻] analyte, compared to the bare electrode, and improved LOD can be achieved by the rGO and CNT-added chitosan modified electrodes, presumably due to the conductive nature of the CNT, platinum black and rGO which account for amplification of the electrochemical signal. Surface roughness of the platinum black and rGO electrodes also contributes by increasing the capacity of the double layer, leading to additive current.

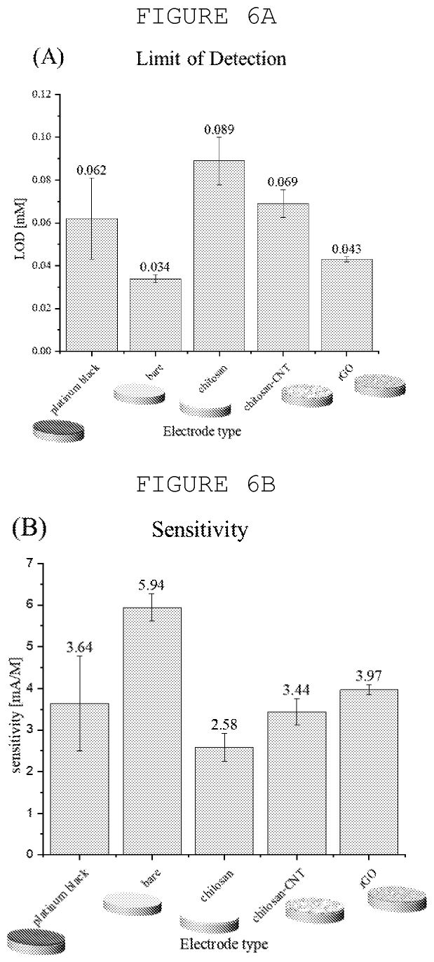

B2: Cyclovoltammetry in solutions of TAC marker (ascorbic acid) and separately in solutions of TOS marker (hydrogen peroxide) was performed across the potential range of −0.1V to +0.65V, for a total of three cycles. Diluted PBS solutions of ascorbic acid or hydrogen peroxide with concentrations spanning the range from 4 mM to 0.125 mM (dilution by factor ½) were prepared. Calibration curves that were generated based on the 0.125, 0.25, 0.5, 1.0, 2.0 and 4.0 mM solutions (current peak versus concentration plots) showed linear relationship, as indicated for the purpose of illustration in FIGS. 5A and 5B, for the signals recorded by the bare electrode in ascorbic acid solutions. The same procedure was applied for all the coated electrodes in respect of both ascorbic acid and hydrogen peroxide, to calculate the sensitivity S_{i,j} and the LOD_{i,j} for electrode i {i=bare, chitosan, chitosan+CNT, Pt and rGO} towards analyte j {ascorbic acid and H₂O₂}.

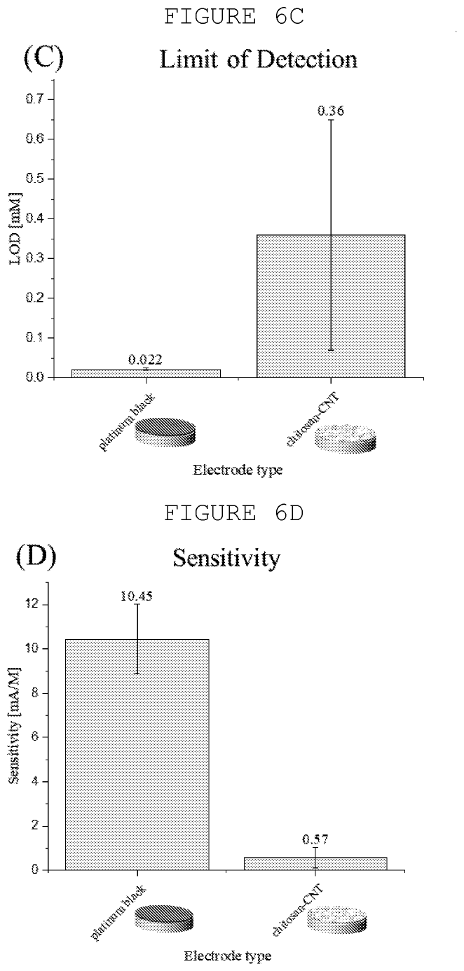

The LOD and sensitivity results are shown in the form of bar diagrams in FIGS. 6A (LOD) and 6B (sensitivity) for i=bare, chitosan, chitosan+CNT, Pt and rGO; j=ascorbic acid} and in FIGS. 6C (LOD) and 6D (sensitivity) for i=chitosan+CNT and Pt; j=hydrogen peroxide). It is seen that all surface-modified electrodes were sensitive towards ascorbic acid with platinum black-coated electrode emerging especially useful in detecting hydrogen peroxide.

Next, three parameters of the entire experimental set-up consisting of n_{bare}=n_{chitosan}=n_{chitosan+CNT}=n_{Pt}=n_{rGO}=2 were calculated. These parameters are:
Sⱼ, which is the average response slope towards analyte j, and is given by:

$${S}_{j} = \frac{1}{n} {\sum }_{i = 1}^{n} {S}_{i , j} \qquad ( 2 )$$

j is ascorbic acid or H₂O₂, S_{i,j} is the corresponding sensitivity as previously defined, and the summation is over the number of electrodes (n=10);
Kⱼ, which is the average signal-to-noise ratio (SNR) in relation to analyte j, and is calculated as follows:

$${K}_{j} = \frac{1}{n} {\sum }_{i = 1}^{n} {K}_{i , j} = \frac{1}{n} {\sum }_{i = 1}^{n} \frac{{s}_{i , j}}{{s}_{i , j}^{2}} \qquad ( 3 )$$

where

$${s}_{i , j}^{2}$$

is the standard deviation of the response slope, and the summation is over the number of electrodes (n=10);
Fⱼ, which is the non-selectivity factor in relation to analyte j, and is defined as:

$${F}_{j} = \frac{{s}_{j}}{{s}_{j}^{2}} \qquad ( 4 )$$

where Sⱼ is the average response slope, as defined in equation (2), and

$${s}_{j}^{2}$$

is its standard deviation.

The results for the two analytes are tabulated in Table 2, indicating the high sensitivity and stability of the electrode array with respect to the tested markers.

TABLE 2

| Analyte | ${S}_{j} = \frac{1}{n} {\sum }_{i = 1}^{n} {S}_{i , j}$ | | ${K}_{j} = \frac{1}{n} {\sum }_{i = 1}^{n} \frac{{S}_{i , j}}{{s}_{i , j}^{2}}$ | ${F}_{j} = \frac{{S}_{j}}{{s}_{j}^{2}}$ |
|---|---|---|---|---|
| Ascorbic acid | 3.9 ± 1.1 | mA/M | 14.8 ± 11.0 | 3.52 |
| H₂O₂ | 3.4 ± 3 | mA/M | 17.0 ± 14.6 | 1.1 |

Next, partial selectivity (PS) was calculated:

$${PS}_{i , j} = \frac{{s}_{i , j}}{{s}_{i , k}} \qquad ( 5 )$$

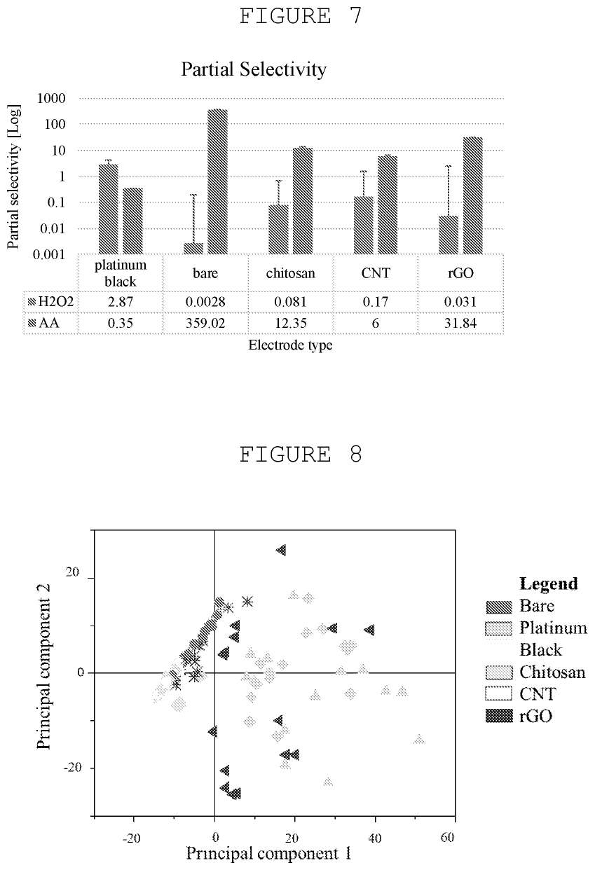

Partial selectivity is calculated by dividing the sensitivity of electrode i towards analyte j (ascorbic acid) by the sensitivity of the same electrode towards the other analyte k (H₂O₂), namely, by dividing the corresponding slopes of the calibration curves. The results are shown graphically in FIG. 7, in the form of bar diagrams, and in tabular form, for each one of the five types of electrodes used, in relation to ascorbic acid and hydrogen peroxide (namely, a pair of bars is assigned to each electrode type, though in fact PS_{i,j} is the inverse of PS_{j,i}). It is seen that the platinum black-coated electrode is unique in that it demonstrates increased partial selectivity towards hydrogen peroxide compared to the other electrodes.

The partial selectivity values were then used to calculate the cross reactivity (CR) over various electrode combinations I, each combination consisting of three electrode types from the group G of five electrode types:

$${CR}_{I , j} = \frac{1}{2} {\sum }_{i ∈ I} {\sum }_{k ∈ I} \vert {PS}_{i , j} - P {S}_{k , j} \vert \qquad ( 6 )$$

The results are tabulated in Table 3:

TABLE 3

| | TAC marker | TOS marker |
|---|---|---|
| electrodes | Ascorbic acid | Hydrogen peroxide |
| chitosan, bare, CNT | 706.05 ± 10.9 | 0.33 ± 2.5 |
| bare, chitosan, rGO | 693.35 ± 11.5 | 0.16 ± 4.7 |
| bare, rGO, CNT | 706.05 ± 11.5 | 0.33 ± 4.7 |
| chitosan, CNT, rGO | 51.7 ± 2.4 | 0.27 ± 3.7 |
| platinum black, chitosan, CNT | 24 ± 1.79 | 5.58 ± 1.57 |
| platinum black, chitosan, rGO | 62.99 ± 2.4 | 5.67 ± 3.7 |
| platinum black, CNT, rGO | 62.99 ± 0.7 | 5.67 ± 2.2 |
| platinum black, bare, CNT | 717.35 ± 11 | 5.73 ± 2.5 |
| platinum black, bare, rGO | 717.35 ± 11.5 | 5.73 ± 4.7 |
| platinum black, bare, chitosan | 717.35 ± 10.9 | 5.73 ± 2.4 |

It may be desired to minimize the size of the electrode array by reducing the number of electrodes employed, because normally only a limited volume of a biofluid sample is available from patients for the tests. The set of operative electrodes may be reduced in number, e.g., three types instead of five, using the cross-reactivity data as a selection criterion. It is seen that useful ternary combinations of electrodes for detecting TAC and TOS markers comprise 1) platinum black-coated electrode and 2) bare electrode, whereas for the third electrode, one may choose from chitosan coated electrode, CNT-added chitosan coated electrode, and reduced graphene oxide-coated electrode, as all three types show roughly comparable contribution to the CR.

### Example 2

Electrochemical Measurement of TAC and TOS Markers in Urine Samples from Healthy Volunteers and Correlation with Spectroscopically Generated Quantification of the Markers

The goal of the study was to correlate between fifteen analyzed urine samples of healthy volunteers to OS parameters measured by the spectroscopic protocols for TAC and TOS quantification.

### Urine Sample Preparation

Urine samples were collected from fifteen healthy volunteers (a volume of 100 ml of the first morning urine). The samples were stored for less than a month in −20 °C. The samples were defrosted at room temperature before the tests. Every sample was divided into duplicates; each duplicate was measured once in random order.

### Electrochemical Measurements

The sample was added to a 20 ml electrochemical cell fitted with a cell cap with the ten-electrode array design shown in FIG. 3 (n_{bare}=n_{chitosan}=n_{chitosan+CNT}=n_{Pt}=n_{rGO}=2). Cyclovoltammetry was performed in urine samples across the potential range of 0.1V to +0.6V, for three cycles, at a scan rate of 0.05 V/s, using the Ivium potentiostat and IviumSoft software.

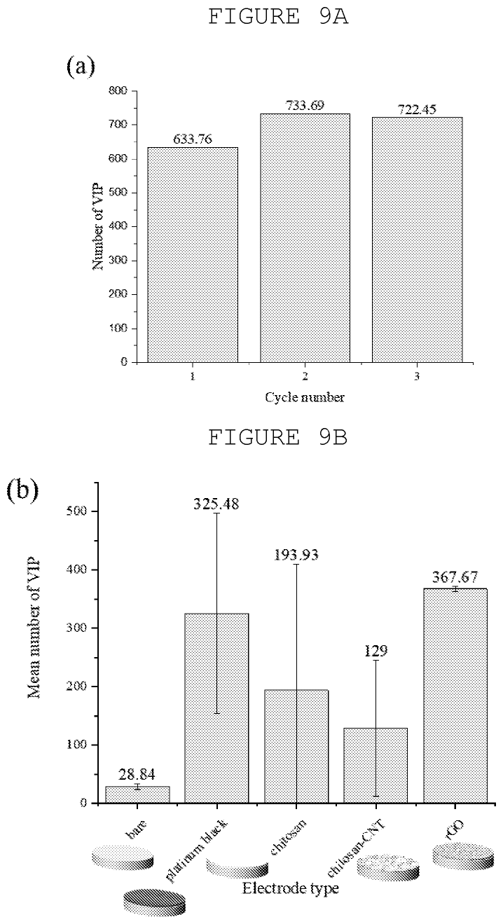

On the same day, oxidative stress was measured by the spectrophotometric technique set out above, and the generated electrochemical signal was inspected accordingly. Principal component analysis was utilized to examine the variability between the coated electrodes. In FIG. 8, the recorded data was projected onto an orthogonal component which correspond to the two most dominant variances. Each of the five colors represents electrode type. For each color, two shapes are used, because there is a pair of electrodes of each type. It is seen that the platinum black and rGO modified electrodes contributed differently to the electrochemical signal, while the bare electrode, the chitosan-coated electrode and the electrode coated with chitosan-CNT showed less variability (the latter two showed similar contribution to the variability of the signal).

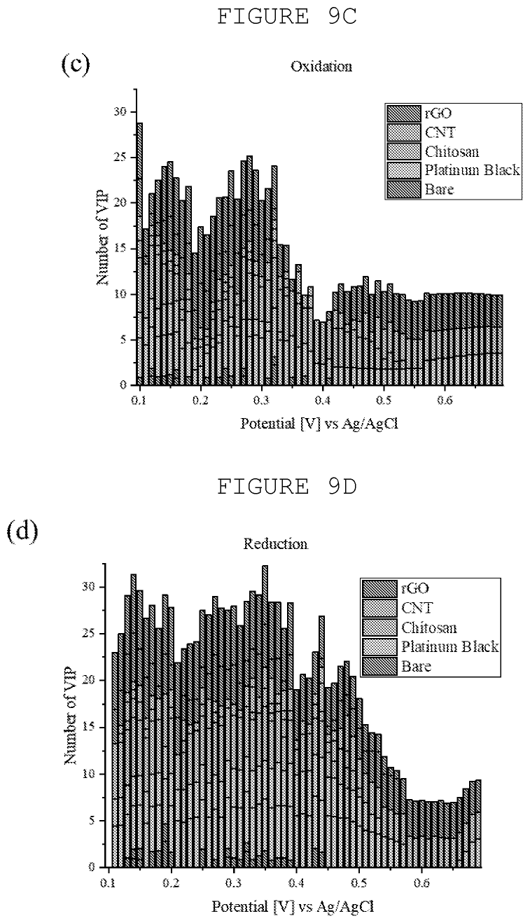

PLSR model was performed to identify the cycle, the electrode and dominant electrochemical signal parameters (such as currents generated at specific potentials) that contributes most to the identification of TAC or TOS concentration. The results are shown in FIG. 9 in the form of bar diagrams in which the abscissa is the number Variable Important in Projection (VIP) and the ordinate is:
the cyclovoltammetry cycle (FIG. 9A, showing three bars for the three cycles);
the electrode (FIG. 9B, showing five bars for the five types of electrodes); and
the potential scanned (oxidation part, FIG. 9C and Reduction part, FIG. 9D).

FIG. 9A suggests that recording two cycles should suffice as the maximal number of VIP was measured for the second cycle. FIG. 9B shows that electrodes coated with platinum black and rGO have the most significant influence on the model (FIG. 9B), perhaps due to the high variability associated with these two electrodes (see FIG. 6). The relation between the weighted VIPs and the potential is seen in FIGS. 9C and 9D. The peak at 0.15 V is assigned to ascorbic acid. At 0.51 V uric acid peak in CNT electrode was observed (FIG. 9C).

### Electrochemical Sensor for Simultaneous Analysis of TAC and TOS

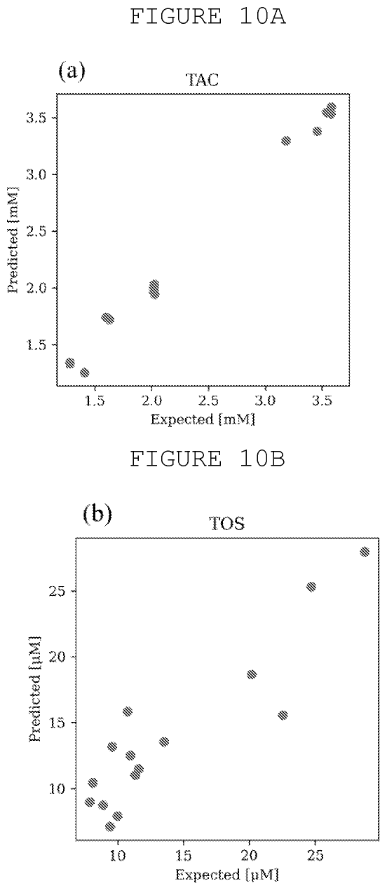

For prediction of the TAC and TOS concentrations new features were generated with python package tsfresh. Grid search with leave-one-out cross-validation was performed on the space of number of features, with 3, 5, 7 and 10 features chosen according to the highest correlation and PCA on the origin data and the extracted features, 32 models in total. The best results of the grid search are 100.4%, 112.47% recovery, 0.99, 0.78 PCC and, 0.12 [mM], 4.9 [μM] RMSE, for TAC and TOS measurements, respectively. FIG. 10 shows the predicted results as a function of the expected for the final model that was trained over all the data, the scores of the model are 100%, 100.8% recovery, 0.99, 0.91 PCC, and 0.08 [mM], 2.7 [μM] RMSE, for TAC and TOS measurements, respectively.

The metrics for regression models are:
Recovery:

$$Recovery = \frac{\tilde{x}}{x} \times 100$$

where \tilde{x} is the estimation of the concentration x.

The Pearson correlation coefficient (PCC) measures the linear relationship between realizations of two random variables:

$$PCC = \frac{\sum ( x - {μ}_{x} ) ( \tilde{x} - {μ}_{\tilde{x}} )}{\sqrt{\sum {( x - {μ}_{x} )}^{2} \sum {( \tilde{x} - {μ}_{\tilde{x}} )}^{2}}}$$

where μₓ is the is the mean of x and μ_{\tilde{x}} is the mean of \tilde{x} The root mean squared error (RMSE): RMSE={square root over (E[(x−\tilde{x})²)])}

1) A method of determining oxidative stress in a subject, comprising:
obtaining a biofluid sample from the subject;
acquiring an electrochemical signal from the sample with the aid of an array of bare and surface-modified electrodes that is sensitive towards a model antioxidant and a model oxidant;
optionally preprocessing the electrochemical signal, to obtain processed data;
applying trained chemometric model(s) to the processed or raw data, to measure TAC and TOS levels; and
determining a status of oxidative stress in the subject based on the measured TAC, TOS or the TOS:TAC ratio.

2) The method according to claim 1, wherein the electrochemical signal is acquired by voltammetry.

3) The method according to claim 1, wherein a urine sample is placed in electrochemical measurement cell equipped with a counter electrode, optionally a reference electrode and an array of working electrodes that comprises at least one working electrode showing sensitivity above 3.0 mA/M for ascorbic acid and at least one working electrode showing sensitivity above 3.0 mA/M for hydrogen peroxide.

4) The method according to claim 3, wherein the array comprises at least one working electrode showing partial selectivity for ascorbic acid/hydrogen peroxide of above 5 and at least one working electrode showing partial selectivity for hydrogen peroxide/ascorbic acid of above 2.

5) The method according to claim 3, wherein the array of working electrodes comprises at least two sets of electrodes selected from:
1) a set consisting of one or more bare electrodes;
2) a set consisting of one or more electrodes coated with polysaccharide film;
3) a set consisting of one or more electrodes coated with polysaccharide film with conductive additives incorporated into the film;
4) a set consisting of one or more electrodes coated with reduced graphene oxide;
5) a set consisting of one or more electrodes coated with platinum black film.

6) The method according to claim 1, wherein the processing of the electrochemical signal comprises one or more of baseline correction, electrode fusion and data normalization.

7) The method according to claim 1, wherein the chemometric model applied is PLSR model that was trained by samples in which TAC and TOS were measured spectroscopically.
